(19) United States
(12) Patent Application Publication
(10) Pub. No.: US 2011/0003870 A1
(43) Pub. Date: Jan. 6, 2011

(54) METHOD OF TREATMENT OF A NEUROLOGICAL DISORDER

(75) Inventors: Michael L. Vieira, Gaithersburg, MD (US); Jones Woodrow Bryan, JR., Gaithersburg, MD (US)

Correspondence Address: Foley & Lardner LLP, 3000 K STREET NW, SUITE 500, WASHINGTON DC, DC, 20007-5109, US

(73) Assignee: Supernus Pharmaceuticals, Inc.

(21) Appl. No.: 12/827,485

(22) Filed: Jun. 30, 2010

Related U.S. Application Data
(60) Provisional application No. 61/222,684, filed on Jul. 2, 2009.

(51) Int. Cl. A61K 31/428; A61P 25/16; A61P 21/00

(52) U.S. Cl. 514/367

(57) ABSTRACT

A method of treating neurological conditions, such as Parkinson's Disease and Restless Leg Syndrome, in a mammalian subject is disclosed. The method comprises the administration of a once-a-day controlled release formulation of pramipexole, which formulation provides a consistently ascending PK profile during sleeping hours.

### CROSS-REFERENCE TO RELATED APPLICATIONS

This application claims priority to U.S. Provisional Application No. 61/222,684, filed Jul. 2, 2009, which is incorporated herein by reference in its entirety.

### BACKGROUND

The invention is directed to a new method of treatment of neurological disorders, e.g., Parkinson's disease, with pramipexole, which is a nonergot dopamine D2/D3 receptor agonist (NEDA). Pramipexole is currently indicated for the treatment of the signs and symptoms of idiopathic Parkinson's disease as well as for the treatment of moderate to severe primary restless leg syndrome (RLS). Pramipexole is commercially available under the tradename Mirapex® (pramipexole dihydrochloride) in an immediate release (IR) tablet at the following doses: 0.125 mg, 0.25 mg, 0.5 mg, 1 mg and 1.5 mg.

Mirapex®, however, exhibits linear pharmacokinetics over its clinical dose range and is rapidly absorbed following oral administration reaching Cₘₐₓ in only about 2 hours. Under the current treatment regimen, therefore, patients are prescribed to take a tablet of Mirapex® three times a day (TID), Even so, patients suffering from neurological diseases, such as Parkinson's disease or RLS, are unable to reap consistent benefit from the medication, or worse, derive little or no benefit from the medication, particularly toward the end of a dose period (e.g., typically in the morning hours).

Because current therapies and traditional administration regimens are not capable of providing relief during, e.g., the sleeping hours and early morning hours, there is a need for a treatment regime that can address this need.

### SUMMARY OF THE INVENTION

The invention provides a novel method of treating neurological conditions in a mammalian subject comprising the administration of a once-a-day controlled release formulation of pramipexole, wherein said administration takes place not earlier than 3 hours before bedtime, and said formulation has a consistently ascending PK profile during the sleeping hours. In one embodiment of the invention, said neurological condition is a Parkinson's disease. In another embodiment of the invention, said neurological condition is restless leg syndrome.

Controlled release formulations of pramipexole useful for the practice of the current invention are not limited to any particular type of controlled release formulations, so long as the formulation is suitable for once a day administration and, being administered at night time, provides consistently ascending plasma levels of pramipexole for the period of time from 6 to 14 hours after ingestion. In a preferred embodiment of the invention, plasma concentration of pramipexole reaches its maximum (Cmax) in the morning or early afternoon hours.

Thus, the controlled release formulation may be in an osmotic formulation, a formulation comprising a release controlling polymer, a formulation comprising a release delaying polymer or combinations thereof.

Further, the controlled release formulation may be presented in a variety of dosage forms well known in the art.

The total amount of pramipexole in the controlled release formulation for the inventive method of treatment may vary from 0.125 mg to 9 mg, but is preferably between 0.5 mg to 6 mg, more preferably between 1.5 mg and 4.5 mg.

### BRIEF DESCRIPTION OF DRAWINGS

FIG. 1 shows simulated steady state plasma profiles for Mirapex® and controlled release formulations of pramipexole administered according to the method of the current invention.

FIG. 2 shows simulated steady state plasma exposures from controlled release formulations of pramipexole administered according to the method of the current invention. These exposures are normalized to those achieved from the same dose of pramipexole given TID in an immediate release formulation.

### DEFINITIONS

For the purposes of this invention, the term “pramipexole” includes pramipexole or any pharmaceutically acceptable salt thereof, as well as any crystalline and non-crystalline form, and any polymorph(s).

An “immediate release formulation” refers to a formulation that releases greater than or equal to 80% of the pharmaceutical agent in less than or equal to about 1 hour.

“Extended release” is defined herein as release of a pharmaceutical agent in a continuous manner over a prolonged period of time.

By “prolonged period of time” is meant a continuous period of time of greater than 8 hours, more preferably greater than 12 hours, more preferably still, greater than 16 hours up to more than 24 hours. “Continuous” is defined as “uninterrupted”.

As used herein, unless otherwise noted, “rate of release” or “release rate” of a drug refers to the quantity of drug released from a dosage form per unit time, e.g., milligrams of drug released per hour (mg/hr) or a percentage of a total drug dose released per hour. Drug release rates for dosage forms are typically measured as an in vitro rate of drug release, i.e., a quantity of drug released from the dosage form per unit time measured under appropriate conditions and in a suitable fluid. The time at which a specified percentage of the drug within a dosage form has been released from said dosage form is referred to as the “Tx” value, where “x” is the percent of drug that has been released.

The release rates referred to herein are determined by placing the dosage form to be tested in an appropriate dissolution media bath. Aliquots of the medium, collected at pre-set intervals, are then injected into a chromatographic system fitted with an appropriate detector to quantify the amounts of drug released during the testing intervals.

“Bedtime” is any period of time wherein sleep for 7 or more hours is intended, and is not necessarily limited to evening or night hours.

“C” denotes the concentration of the drug in blood plasma, or serum, of a subject, and is generally expressed as mass per unit volume, for example, nanograms per milliliter (ng/ml). For convenience, this concentration may be referred to herein as “drug plasma concentration,” “plasma drug concentration” or “plasma concentration” which is intended to be inclusive of a drug concentration measured in any appropriate body fluid or tissue. The plasma drug concentration at any time following drug administration is referenced as Ctime, as in C9 hr or C4 hr, etc.

The maximum plasma drug concentration during the dosing period is referenced as Cmax, while Cmin refers to the minimum blood plasma drug concentration at the end of a dosing interval; and Cave refers to an average concentration during the dosing interval.

The “percent of fluctuation” for a dosing period is defined as a quotient (Cmax−Cmin)/Cave*100%.

Persons of skill in the art will appreciate that plasma drug concentrations obtained in individual subjects will vary due to inter-patient variability in the many parameters affecting drug absorption, distribution, metabolism and excretion. For this reason, unless otherwise indicated, when a drug plasma concentration is listed, the value listed is the calculated mean value based on values obtained from a group of subjects tested.

The term “bioavailability” refers to an extent to which, and sometimes the rate at which, the active moiety (drug or metabolite) enters the systemic circulation, thereby gaining access to the site of action.

“AUC” is the area under the plasma concentration-time curve and is considered to be the most reliable measure of bioavailability. The AUC is directly proportional to the total amount of unchanged drug that reaches the systemic circulation. AUC is a measure of exposure of the body to the drug.

Side effect is defined herein as any undesirable secondary, usually adverse, effect of a drug.

For the purposes of this application, two formulations are given in the “equivalent amount” if they produce an AUC within 80% to 125% of each other for the same period of time.

Throughout this application, “administered tid” means that ⅓ of the total daily dose of the active agent is to be administered every 8 hours.

Unless otherwise specified, “a” or “an” means “one or more”.

### DETAILED DESCRIPTION OF THE INVENTION

The inventive method of treatment is designed, in part, to address problems associated with the day-time administration of pramipexole, such as interrupted sleep patterns, resultant fatigue, need for more “therapy” and the need for better motor functionality during waking hours. The inventive method, which comprises dosing pramipexole formulation late in the day, allows for rising/ascending PK profiles through sleeping hours and into the following afternoon.

This new dosing paradigm offers higher and ascending drug exposure during early morning hours to enable and improve motor functionality at time of waking and through early part of the day. Additionally the new dosing paradigm and associated higher/ascending plasma levels may enable more continuous sleep (due to blunted Cmax vs pramipexole IR) in the case of PD or RLS thus translating into less daytime fatigue as has been reported by Mirapex® users.

Controlled release formulations suitable for the inventive method are preferably designed in such a way that pramipexole is released from the formulation along a pre-determined release profile. Exemplary formulations are discussed hereinbelow and may also be found in U.S. patent application Ser. No. 12/478,979, filed Jun. 5, 2009, the disclosure of which is incorporated herein in its entirety by reference. In one embodiment, a once a day administration of the formulation of the current invention results in the bioavailability that is equivalent to that produced by the equivalent amount of pramipexole administered as an immediate release formulation TID.

In another embodiment of the invention, the pre-determined release profile of the inventive formulation is such that a maximum steady state plasma concentration (Cmax) of pramipexole is not higher than the maximum plasma concentration produced by the equivalent amount of pramipexole administered as an immediate release formulation TID, and a minimum steady state plasma concentration (Cmin) is not lower than 75% of the minimum plasma concentration produced by the equivalent amount of pramipexole administered as an equivalent immediate release formulation TID.

In yet another embodiment, the profile is such that the degree of fluctuation is in the range of from 50% to 125% of the degree of fluctuation produced by the equivalent amount of pramipexole administered as an immediate release formulation TID.

The current invention comprises a formulation of pramipexole such that at least 80% of the active ingredient is released in a time period of from 12 to 24 hours, and preferably, in a time period of from 12 to 14 hours. Alternatively, the formulation may be designed in a way that at least 80% of the active ingredient is released at the time period of from 16 to 18 hours. In a further embodiment, at least 80% of the active ingredient is released at the time period of from 20 to 24 hours.

In one embodiment of the invention, the controlled release formulation is an osmotic formulation comprising a therapeutically effective amount of pramipexole, an osmotic agent and a semipermeable membrane.

In another embodiment of the invention, a controlled release formulation comprises a release delaying polymer selected from the group consisting of Eudragit FS 30 D (poly (methyl acrylate-co-methyl methacrylate-co-methacrylic acid)), Eudragit L and S (poly (methacrylic acid-co-methyl methacrylate)) hydroxypropyl methylcellulose phthalate, hydroxypropyl methylcellulose acetate succinate, cellulose acetate phthalate, cellulose acetate trimelliate, polyvinyl acetate phthalate, shellac, and zein; an extended release polymer selected from a group consisting of cellulose acetate, cellulose acetate butyrate, cellulose acetate propionate and derivatives thereof, cellulose acylate, ethylcellulose, polyvinyl acetate, Eudragit NE 30 D poly(ethyl acrylate-co-methyl methacrylate), Eudragit RS and RL poly (ethyl acrylate-co-methyl methacrylate-cotrimethylammonioethyl methacrylate chloride) ammonio methacrylate copolymer Type B NF and ethyl acrylate methyl methacrylate copolymer, and combinations thereof.

In yet another embodiment of the invention, the controlled release formulation comprises at least one extended release component and at least one immediate release component.

Controlled release formulations of the current invention may comprise more than one extended release component, each characterized by its own release profile, or a combination of at least one extended release component and a delayed release component. Indeed, the inventive method of the current invention contemplates for the flexible use of slightly different controlled release formulations to provide a release profile and drug exposure uniquely suitable for the needs of each individual patient.

The invention is further illustrated by, though in no way limited to, the following examples.

### EXAMPLES

### Example 1

The following table provides non-limiting examples for three formulations of Pramipexole XR (i.e., Tablet A, Tablet B and Tablet C).

TABLE 1

| | | Tablet A | | Tablet B | | Tablet C | |
|---|---|---|---|---|---|---|---|
| | | Quantity | | Quantity | | Quantity | |
| Ingredient | Function | (mg) | % (w/w) | (mg) | % (w/w) | (mg) | % (w/w) |
| Pramipexole | Drug | 0.75 | 0.36 | 0.75 | 0.36 | 0.75 | 0.36 |
| Dihydrochloride | | | | | | | |
| Monohydrate^{a} | | | | | | | |
| Mannitol, USP | Osmotic | 103.80 | 49.90 | 103.80 | 49.71 | 103.80 | 49.24 |
| | agent | | | | | | |
| Isomalt, NF | Osmotic | 86.78 | 41.72 | 86.78 | 41.56 | 86.78 | 41.17 |
| | agent | | | | | | |
| Povidone, USP | Binder | 6.67 | 3.21 | 6.67 | 3.19 | 6.67 | 3.16 |
| Magnesium | Lubricant | 2.00 | 0.96 | 2.00 | 0.96 | 2.00 | 0.95 |
| Stearate, NF^{b} | | | | | | | |
| Cellulose | Extended | 6.40 | 3.08 | 7.04 | 3.37 | 8.64 | 4.10 |
| Acetate, NF | Release | | | | | | |
| Triethyl Citrate, | Plasticizer | 1.60 | 0.77 | 1.76 | 0.84 | 2.16 | 1.02 |
| NF | | | | | | | |
| | Total | 208.0 | 100 | 208.8 | 100 | 210.8 | 100 |

^{a}The drug products are formulated to provide the pramipexole dose strength consistent with the commercially available immediate release tablet formulations, Mirapex ® (pramipexole dihydrochloride tablets). The “label dose” strength of Mirapex ® tablets is based on the drug substance form pramipexole dihydrochloride monohydrate.
^{b}Vegetable origin

Pharmacokinetic parameters for the formulations of Example 1 are as follows:

TABLE 2

| | Tablet A | Tablet B | Tablet C | Mirapex |
|---|---|---|---|---|
| Total Dose (mg) | 0.75 | 0.75 | 0.75 | 0.75 |
| Cₘₐₓ (pg/mL) | 688.4 | 613.4 | 549.4 | 669.5 |
| tₘₐₓ (hr) | 16.0 | 16.0 | 16.0 | 18.0 |
| t_{1/2} (hr) | 9.9 | 10.0 | 9.7 | 9.4 |
| AUC₀₋₂₄ (pg hr/mL) | 11300.4 | 9951.8 | 8172.7 | 11792.7 |
| AUCₗₐₛₜ (pg hr/mL) | 18762.8 | 18421.4 | 16646.0 | 17636.7 |
| AUC_{inf} (pg hr/mL) | 18840.3 | 18712.8 | 16907.6 | 18045.6 |
| Rel BA (AUC₀₋₂₄) | 0.96 | 0.84 | 0.69 | NA |
| Rel BA (AUCₗₐₛₜ) | 1.06 | 1.04 | 0.94 | NA |
| Rel BA (AUC_{inf}) | 1.04 | 1.04 | 0.94 | NA |

### Example 2

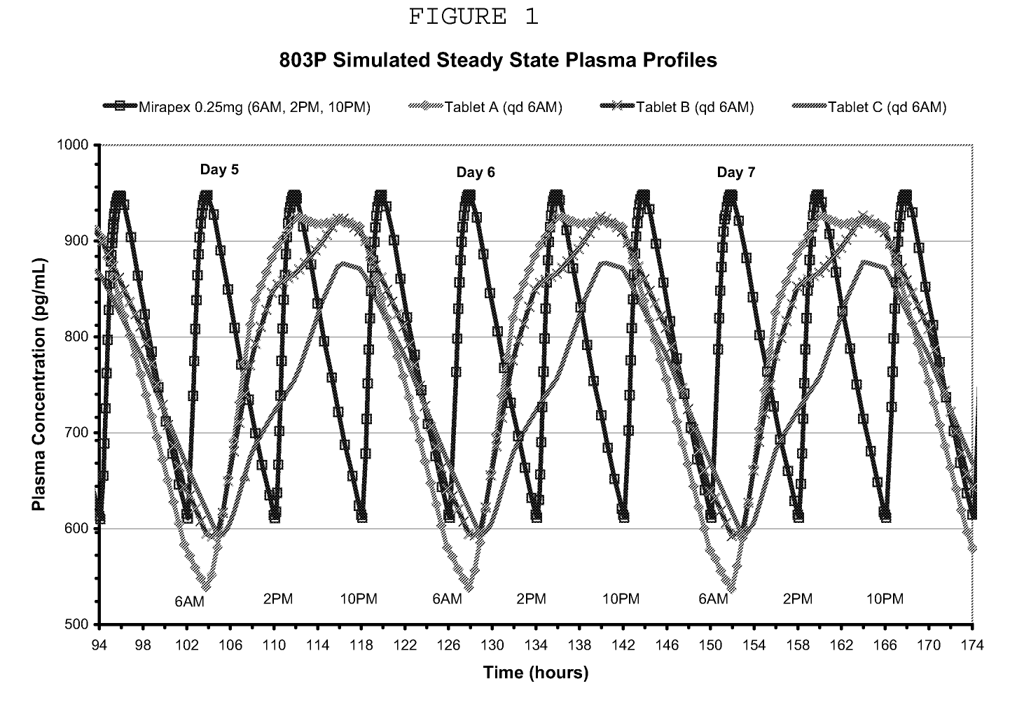

Pharmacokinetic parameters of Table 2 were used as a basis for the in-silico steady state plasma profile simulations for Tablets A, B, and C. The results of the simulation are represented in Table 3 and FIG. 1. WinNonlin® version 5.0.1 and 5.2 (Pharsight Corporation Mountain View, Calif. 94041) and GastroPlus™ version 5.3 and 6.0 (Simulations Plus, Inc. West Lancaster, Calif. 93534) were used to perform the in-silico simulations.

TABLE 3
Steady State Simulations

| | Tablet A | Tablet B | Tablet C | Mirapex |
|---|---|---|---|---|
| Total Dose (mg) | 0.75 | 0.75 | 0.75 | 0.75 |
| Cmax (pg/mL) | 926 | 926.2 | 877.8 | 983.5 |
| Cmin (pg/mL) | 536.9 | 592.3 | 592.1 | 603.3 |
| Cave (pg/mL) | 785.3 | 791.5 | 745 | 797.1 |
| AUC144 hr-168 hr | 18847.3 | 18661.9 | 17880.3 | 19130 |
| Fluctuation (%) | 49.5 | 42.2 | 38.3 | 47.7 |
| Relative BA | 0.99 | 0.98 | 0.93 | NA |
| AUC144 hr-168 hr | | | | |

### Example 3

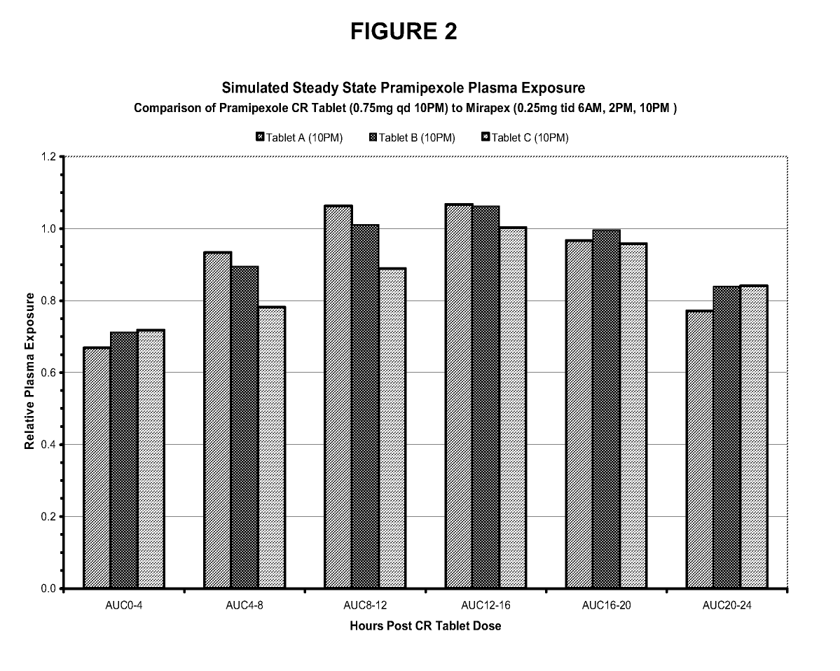

Controlled release formulation A of Example 1 characterized by T80 of about 12-14 hours is administered to a patient at 10 pm in the total amount equivalent to the TID administration of the immediate release formulation. The peak plasma exposure of pramipexole is reached by 6 AM and is maintained till 2 PM, thus providing the patient with the adequate pharmacological support during the waking, morning, and early afternoon hours (FIG. 2).

### Example 4

Controlled release formulation C of the Example 1, characterized by T80 of about 20 hours is administered to the patient at 10 pm in the total amount equivalent to the TID administration of the immediate release formulation. The peak plasma exposure of pramipexole is reached by 10 AM and is maintained till 6 PM thus providing the patient with the adequate pharmacological support during the late morning and afternoon hours (FIG. 2).

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A method of treating Parkinson's disease in a mammalian subject comprising once-a-day administration of a controlled release formulation of pramipexole, wherein said administration takes place not earlier that 3 hours before bedtime, and said formulation exhibits a consistently ascending PK profile during the sleeping hours.

2. The method of claim 1, wherein said administration results in improved motor functionality or less fatigue during the morning and early afternoon hours.

3. The method of claim 1, wherein Cmax is reached during morning or early afternoon hours.

4. The method of claim 1, wherein said controlled release formulation is an osmotic formulation.

5. The method of claim 1 wherein said formulation comprises at least one polymer selected from a extended release polymer, release-delaying polymer or a combination thereof.

6. The method of claim 1 resulting in the improved physical activity of the subject during the morning hours.

7. A method of treating restless leg syndrome in a mammalian subject comprising once-a-day administration of a controlled release formulation of pramipexole, wherein said administration takes place not earlier that 3 hours before bedtime, and said formulation exhibits a consistently ascending PK profile during the sleeping hours.

8. The method of claim 1, wherein said administration results in improved motor functionality or less fatigue during the morning and early afternoon hours.

9. The method of claim 1, wherein Cmax is reached during morning or early afternoon hours.

10. The method of claim 1, wherein said controlled release formulation is an osmotic formulation.

11. The method of claim 1 wherein said formulation comprises at least one polymer selected from a extended release polymer, release-delaying polymer or a combination thereof.

12. The method of claim 1 resulting in the improved physical activity of the subject during the morning hours.
